(19) United States
(12) Patent Application Publication
(10) Pub. No.: US 2008/0003244 A1
(43) Pub. Date: Jan. 3, 2008

(54) Inflammation and Oxidative Stress Reducing Compostion for Topical or Oral Administration

(76) Inventor: Gary Viole, Ridgewood, NJ (US)

Correspondence Address: COOPER & DUNHAM, LLP, 1185 AVENUE OF THE AMERICAS, NEW YORK, NY, 10036, US

(21) Appl. No.: 11/770,390

(22) Filed: Jun. 28, 2007

Related U.S. Application Data
(60) Provisional application No. 60/817,326, filed on Jun. 28, 2006.
(60) Provisional application No. 60/850,866, filed on Oct. 10, 2006.

(51) Int. Cl. A61K 31/685; A61K 31/385; A61K 9/00

(52) U.S. Cl. 424/400; 424/445; 514/78; 514/440

(57) ABSTRACT

The invention provides, in one embodiment, a method for making lecithin phospholipid with lipoic or piperic acid based composition, a preparation for use as a topical skin application for treatment of infectious skin lesions such as acne, for the protection of skin cells from oxidative agents, and as anti-inflammatory treatments.

### CROSS-REFERENCE

This application claims priority of United States Provisional Application Patent (U.S. Ser. No. 60/817,326, filed Jun. 28, 2006), and United States Provisional Patent Application (U.S. Ser. No. 60/850,866, filed Oct. 10, 2006). The contents of both provisional applications are incorporated herein by reference.

### FIELD OF THE INVENTION

This invention relates to soy lecithin derived compositions useful for oral administration to protect cells from oxidative stress from endogenous and exogenous sources, and to such compositions useful as wound covering membranes and as wound glues or adhesives.

### BACKGROUND OF THE INVENTION

Cellular oxidative stress from the production of reactive oxidative species [ROS] produced by cells during normal metabolic processes and external oxidative agents result in cumulative cellular damage, DNA damage and inflammatory response. This damage is associated with the aging process as well as most chronic progressive disorders such as heart disease, diabetes, neurological disorders and cancer. Strategies to manage ROS including exogenous anti-oxidants and genetic manipulations to cause cells to over-express endogenous anti-oxidants appear to have met with mixed success.

In addition, others have experimented with various types of membranes to cover wounds. For example, U.S. Pat. No. 4,703,108 (Silver) discloses a biodegradable matrix made of a form of collagen that can be used in sponge or sheet form as a synthetic skin for topical use with external or internal wounds.

Wound adhesives have also enjoyed increasing popularity as an aid to healing for surgical incisions or accidentally induced wounds. For example, U.S. Pat. No. 6,939,365 (Stoltz) discloses a composite tissue adhesive derived from collagen that helps to adhere tissue or fill tissue space. (The foregoing patents and all others cited herein are incorporated by reference.)

It would be desirable to provide compositions based upon phospholipids that reduce reactive oxidative stress in patients, and that could be used as wound coverings and adhesives.

### SUMMARY OF THE INVENTION

The invention provides, in one embodiment, a method for making lecithin phospholipid derivatives, a preparation for use as a topical skin application for treatment of infectious skin lesions such as acne, for the protection of skin cells from oxidative agents, and as anti-inflammatory treatments.

The invention further provides a method for making preparations for oral administration that provide systemic oxidative stress protection. The method comprises adding lecithin or other similar phospholipid to an alcohol or an alcohol based water solvent mixture, and then adding lipoic acid, piperic acid or other acid, bringing the mixture to a boil. After that, the first insoluble fraction is separated from the first soluble fraction. The first soluble fraction is then cooled and separated from a second insoluble fraction. The second soluble fraction has solvent removed to obtain a usable product, or the two insoluble fractions obtained above are added to aqueous alcohol with lipoic acid or another acid and heated to a boil. The liquid fraction is separated and then allowed to cool or is cooled, following which the insoluble fraction is removed. The two soluble fractions obtained after cooling are then combined. Solvent is then removed from the combined fractions to obtain the usable product.

The invention provides, in another embodiment, a membrane made from the foregoing composition for use in covering and protecting wounds, derived from soy derived phospholipids.

The invention also provides a method for making a surgical glue from the composition above.

### DETAILED DESCRIPTION OF THE PREFERRED EMBODIMENTS

In one embodiment, the invention provides a method of producing a composition from soy lecithin and lipoic, piperic, or other acid comprising heating lecithin in a water and alcohol mixture until about boiling, adding an amount of lipoic or piperic acid sufficient to create first soluble and insoluble fractions; removing the first insoluble fraction from the first soluble fraction; adding a water and alcohol mixture to the first insoluble fraction and heating until about boiling; cooling the mixture and then separating a second soluble fraction from a second insoluble fraction; combining the first and second liquid fractions; and removing solvent from combined first and second fractions to yield a lecithin/lipoic or piperic acid composition.

The invention further provides a method for preventing oxidative stress to an area of skin on a patient in need of such treatment comprising applying to the skin an amount of the lecithin/lipoic or piperic acid composition in a pharmaceutically acceptable vehicle effective to prevent oxidative stress to the skin on which the composition is applied. In one preferred use, the skin is undergoing radiation therapy.

The invention also provides a method for reducing inflammation in skin and adjacent tissues in a patient comprising applying an amount of the lecithin/lipoic or piperic acid composition in a pharmaceutically acceptable vehicle effective to reduce inflammation to the skin and adjacent tissues to which the composition is applied.

The invention additional provides a method for reducing microbial growth on skin comprising applying an amount of the lecithin/lipoic or piperic acid composition in a pharmaceutically acceptable vehicle effective to reduce microbial growth on skin to which the mixture is applied.

In another embodiment, the invention provides a method for increasing susceptibility of cancer cells to treatment by radiation therapy or chemotherapy to a patient undergoing such treatment, comprising administering topically or orally to the patient an amount of the lecithin/lipoic or piperic acid composition in a pharmaceutically acceptable vehicle effective to increase susceptibility of cancerous cells in the patient to chemotherapy or radiation therapy. Preferably, the composition is administered orally.

The invention provides in a further embodiment a method of protecting cells from endogenous and exogenous oxidative stress damage in a patient comprising administering to the patient an effective amount of the lecithin/lipoic or piperic composition.

In yet another embodiment, the lecithin/lipoic or piperic composition is applied to a wound and forms a protective membrane or wound covering thereon. The lecithin/lipoic or piperic acid composition may also be applied to a wound as an adhesive or wound glue.

The phospholipid source is preferably soy derived lecithin, but may be lecithin derived from egg or another source, or may be a phospholipid of similar structure to lecithin.

The solvent used to create the lecithin/lipoic or piperic composition may be any organic or aqueous solvent that can dissolve both the lecithin phospholipid acid. Preferably, a non-toxic solvent or solvent mixture is used such as ethye and lipoic or piperic alcohol and water to avoid the necessity of removing possibly harmful or toxic solvents from the resulting composition.

As an acid for use in making the lecithin or phospholipid based active ingredient, lipoic acid, piperic acid or other similar acid can be used with the lecithin phospholipid.

For topical administration the gel-like composition can be applied as is (without further dilution or compounding), or it can be admixed with suitable pharmaceutically acceptable carriers or vehicles, as set forth below.

For oral administration, the composition in the form of an oil or gel is preferably mixed with glycerin, aloe vera, water, and lemon oil. The dosage of phospholipid/lipoic active ingredient is about 35 to 40%, diluted to make it palatable. About 6 to 8 grams a day appears to be a desired therapeutic dose, although it can vary from 5 grams to 20 or more grams of composition by weight depending on the purpose for which it is being administered.

The following delivery systems, which employ a number of routinely used acceptable pharmaceutical carriers or vehicles, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprolactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g. propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

The following examples are illustrative of the preferred embodiments of the present invention, and should not be regarded as limiting.

### EXAMPLE 1

Commercial granular lecithin 120 grams combined with 250 ml 95% ethanol-5% water and 6.25 grams lipoic acid are brought to a boil, removed from the heat, and the liquid fraction is separated from the insoluble fraction. The liquid fraction is cooled, preferably in a freezer, to 35 degrees C. The soluble fraction is then separated from a second insoluble fraction. To recover the extract product, LP 0817, ethanol is evaporated from the soluble fraction under vacuum. A LP 0817 is mixed with water, glycerin, pantethine or other suitable carrier to obtain a useable form.

Variations of this procedure include increasing the quantity of lipoic acid or similar chemical to as high as 25 grams for the extract process and forming mixtures with combinations of glycerin, pantethine or water. This can result in improvements in certain applications, i.e. 30% extract with 12.5 grams lipoic acid, 30% pantethine and 40% glycerin seems to have enhanced anti-microbial and anti-inflammatory properties. A mixture of LP 0817 30% by weight and water 70% by weight has tested positive for oxidative stress protection for cells in vitro. Implications for topical use include to protect skin from age-related damage due to exogenous and endogenous oxidative stress; to prevent skin cancer due to UV sunlight exposure; and to protect tissue from damage due to exposure to ionizing radiation treatment. The composition is also anti-inflammatory and anti-microbial.

When ingested orally, alone or in mixture, the composition imparts systemic protection against oxidative stress, which may make it useful in combating the effects of aging, heart disease, diabetes, neurological disorders, arthritis, and general chronic progressive disorders. With cancer cells, an improvement in treatment sensitivity is noted, as resistance to treatment is associated with oxidative species production by cancer cells. In other words, the compositions of the present invention can increase the susceptibility of cancer cells to treatments including radiation and chemotherapy, while also reducing oxidative stress to the healthy cells, tissues, and organs of the patient undergoing treatment for cancer.

### EXAMPLE 2

Preferred oral dose preparation—120 grams of commercial granular lecithin is combined with 6.25 to 25 grams of lipoic acid, piperic acid, or another acid, and 250 to 320 ml of a 90% ethanol-10% water solution. The combination is brought to a boil, removed from the heat, and the liquid fraction is separated from the insoluble fraction. The soluble liquid fraction is cooled in a freezer to 33 degrees C., and further separated from insolubles. Both insoluble fractions are combined with 90 ml of an 80% ethanol-20% water solution and 1 to 4 grams lipoic acid or similar composition and brought to a boil. The liquid soluble fraction is separated and cooled in a freezer to 42 degrees C. Both liquid soluble fractions are combined and the ethanol/water solvent is removed under vacuum to recover the extract product.

The foregoing composition may be administered as an agent to prevent or reduce oxidative stress. It can impart systemic protection against oxidative stress caused by a number of factors including aging, heart disease, diabetes, neurological disorders, arthritis, and other generic chronic progressive disorders. With patients having cancer, oral administration of the composition can increase effectiveness of chemotherapeutic agents and radiation against cancer cells, while working against the oxidative species produced by cancer cells.

### EXAMPLE 3

An in vitro assay was done to demonstrate the oxidative stress protection imparted to cells by the phospholipid-lipoic acid composition is unique to the composition and is absent when the components are tested independently.

A549 human lung epithelial cells were seeded and treated with 2.5 mM H202 with or without lipid for 4 hours. Cells were washed and trypsinized and live cells were counted.

Test Sample Composition—The extract product titled LP 0817, example 1, was mixed with water at a 30% concentration yielding a compostion of Lipid Extract 23%, Lipoic Acid 7%, Water 70%.

| | Lipid conc. | | | |
|---|---|---|---|---|
| | 5% + H2O2 | 2% + H2O2 | 1% + H2O2 | 0% + H2O2 |
| Cell Number | 15 | 7 | 56 | 10 |
| #081706 | | | | |
| Cell Number | 13 | 13 | 40 | 10 |
| #090506 | | | | |

### SUMMARY

Both samples #081706 and #090506 protected cells from treatment of H202. The optimal concentration was also 1%. Cells pinocytosed lipid in the cell body and cells look vacuolated. Compared to H202 positive control, cells were not dying by apoptosis.

### EXAMPLE 4

Lipid extract (produced by alcohol extraction process cited in example 1 without lipoic acid component)—23% lipids/77% water and lipoic acid at 7% concentration tested separately.

210 mg lipoic acid was dissolved with 2.8 ml. DMSO. (93% DMSO and 7% Lipoic Acid.) This is 100% solution. This working solution was diluted to 5%, 2%, 1% to use for hydrogen peroxide study.

A549 human lung epithelial cells (1.5×10⁵/well in 6 well dish) were seeded and treated with 2.5 mM H202 with or without lipid for 4 hours. Cells were washed and trypsinized and live cells (trypan blue dye exclusion assay) were counted. Cell number is represented as 10⁴.

Lipid extracts 23%, water 77%

| | Lipid conc. | | | | |
|---|---|---|---|---|---|
| | | | | 5% + | w/o |
| | 0% + H2O2 | 1% + H2O2 | 2% + H2O2 | H2O2 | H2O2 |
| Live cell | 12 | 16 | 8 | 10 | 58 |
| Number | | | | | |
| Live cell | 11 | 13 | 12 | 12 | 53 |
| Number | | | | | |

Lipoic Acid 7%

| | | | | 5% + | w/o |
|---|---|---|---|---|---|
| | 0% + H2O2 | 1% + H2O2 | 2% + H2O2 | H2O2 | H2O2 |
| Live cell | 12 | 13 | 14 | 10 | 58 |
| Number | | | | | |
| Live cell | 11 | 14 | 14 | 12 | 53 |
| Number | | | | | |

### CONCLUSION

Neither Lipid extract nor Lipoic acid protected the cells from H202 damage. Cell morphology changed with H202 treatment. Cells became flat. With trypsinization, cells did not round up like control cells.

### EXAMPLE 5

120 grams of soy lecithin is added to 250 ml 95% ethyl alcohol. The mixture is brought to a boil then allowed to cool until the boiling has stopped. 25 grams of lipoic acid is added to the mixture, and the liquid fraction is separated from the insoluble fraction. The liquid fraction is then cooled to produce a clear solution that is separated from any residual insolubles, and is labeled Product A.

To the insoluble fraction remaining from the production of Product A above, 120 ml of a solution of 20% water, 80% ethyl alcohol is added and the mixture is brought to a boil. The mixture is then allowed to cool to the point where the boiling has stopped. The liquid fraction is then separated from the insoluble fraction, and added to the Product A solution. The resulting solution is then allowed to cool to a clear solution and separated from any residual insolubles.

At this point, the alcohol and water are removed leaving behind a material that is elastic, flexible, durable, anti-microbial and highly water resistant, identified as Product B. Product B can be dried to form a membrane that is suitable for wound covering in that it is bio-compatible and will form a barrier against infection as well as keep the wound bed moist to aid healing.

### EXAMPLE 6

120 grams of soy lecithin is added to 250 ml 95% ethyl alcohol. The mixture is brought to a boil then allowed to cool until the boiling has stopped. 25 grams of lipoic acid is added to the mixture, and the liquid fraction is separated from the insoluble fraction. The liquid fraction is then cooled to produce a clear solution that is separated from any residual insolubles, and is labeled Product C.

At this point, the alcohol can be removed from Product C leaving behind a viscous, oily substance that is highly water resistant, anti-microbial and may have use in wound dressing preparation or to close incision wounds.

### EXAMPLE 7

The viscous, oily substance obtained in Example 6 above was mixed with about 60 to 70 percent glycerin. This mixture was dabbed on active acne lesions in a volunteer subject, and noticeable improvement occurred in the size and redness of the lesions after several hours. The next day further improvement of lesions was noticed. The composition has apparent antimicrobial properties, and has potential as a penetration enhancer for other active ingredients.

I claim:

1. A method of producing a composition from lecithin and lipoic, piperic, or other acids, comprising:
heating lecithin in a water and alcohol mixture until about boiling;
adding an amount of lipoic or piperic acid sufficient to create first soluble and insoluble fractions;
removing the first insoluble fraction from the first soluble fraction;
adding a water and alcohol mixture to the first insoluble fraction and heating until about boiling;
cooling the mixture and then separating a second soluble fraction from a second insoluble fraction;
combining the first and second liquid fractions; and
removing solvent from combined first and second fractions to yield a lecithin/lipoic or piperic acid composition.

2. A method for preventing oxidative stress to an area of skin to a patient in need of such treatment comprising:
applying to the skin an amount of the lecithin/lipoic or piperic acid composition of claim 1 effective to prevent oxidative stress in the area of skin on which the composition is applied.

3. A method in accordance with claim 1, wherein the skin is exposed to radiation from radiation therapy.

4. A method for reducing inflammation in skin and adjacent tissues in a patient, comprising:
applying an amount of the lecithin/lipoic acid or piperic acid composition of claim 1 effective to reduce inflammation in the skin and adjacent tissues to which the composition is applied.

5. A method for reducing microbial growth on skin, comprising:
applying an amount of the lecithin/lipoic or piperic acid composition of claim 1 effective to reduce microbial growth on skin to which the mixture is applied.

6. A method for increasing susceptibility of cancer cells to radiation or chemotherapy treatment in a patient undergoing such treatment, comprising:
administering topically or orally to the patient an amount of the lecithin/lipoic or piperic acid composition of claim 1 effective to increase susceptibility of cancerous cells in the patient undergoing chemotherapy or radiation therapy.

7. A method according to claim 6, wherein the composition is administered orally in a pharmaceutically acceptable vehicle.

8. A method according to claim 6, wherein the composition is administered topically in a pharmaceutically acceptable vehicle.

9. A method of protecting cells from endogenous and exogenous oxidative stress damage in a patient comprising administering to the patient an effective amount of the composition of claim 1.

10. A method according to claim 1, wherein the lecithin/lipoic or piperic composition is applied to a wound and forms a protective membrane thereon.

11. A wound covering made in accordance with the method of claim 10.

12. A method according to claim 1, wherein the lecithin/lipoic or piperic acid composition is applied to a wound as wound adhesive or wound glue.

13. A wound glue or surgical adhesive made in accordance with the method of claim 12.
